(19) United States
(12) Patent Application Publication
(10) Pub. No.: US 2010/0292321 A1
(43) Pub. Date: Nov. 18, 2010

(54) BIOFILM CONTROL

(75) Inventors: Joe D. Sauer, Baton Rouge, LA (US); Christopher J. Nalepa, Baton Rouge, LA (US); William S. Pickrell, Motoazabu (JP); Richmond M. Starrett, Baton Rouge, LA (US); Hassan Y. Elnagar, Baton Rouge, LA (US); George W. Cook, Baton Rouge, LA (US); Robert M. Moore, Baton Rouge, LA (US); Jennifer Moore, Baton Rouge, LA (US)

Correspondence Address: ALBEMARLE CORPORATION;PATENT DEPARTMENT, 451 FLORIDA STREET, BATON ROUGE, LA, 70801, US

(73) Assignee: ALBEMARLE CORPORATION, BATON ROUGE, LA (US)

(21) Appl. No.: 12/300,367

(22) PCT Filed: Jun. 29, 2007
(86) PCT No.: PCT/US07/72516

§ 371 Date: Oct. 6, 2009

Related U.S. Application Data
(60) Provisional application No. 60/817,384, filed on Jun. 29, 2006.

(51) Int. Cl. A01N 43/08; C12N 1/00

(52) U.S. Cl. 514/472; 435/243

(57) ABSTRACT

Compositions and methods suitable for killing bacteria and controlling biofilms comprising one or more microorganisms are provided wherein molecules capable of emulating cell-to-cell signal molecules of the microorganisms are utilized.

### BACKGROUND

Biofilms are mucilaginous communities of microorganisms such as bacteria, archaea, fungi, molds, algae or protozoa or mixtures thereof that grow on various surfaces. Biofilms form when microorganisms establish themselves on a surface and activate genes involved in producing a matrix that includes polysaccharides. This matrix may provide protection of biofilm bacteria from biocides.

Molecules called quorum-sensing signals help trigger and coordinate part of the process of forming a biofilm via cell-to-cell communication/signalling. Bacteria constantly secrete low levels of the signals and sense them either through receptors on their surfaces, or internally. The receptors trigger behavioral changes when there are enough bacteria to allow the signals' concentrations to achieve a critical threshold. Once this occurs, bacteria respond by adopting communal behavior, such as forming a biofilm, and in the case of pathogenic bacteria, deploying virulence factors such as toxins. In addition to communicating with members of their own species, bacteria also conduct inter-species communications, such that a biofilm may contain more than one species of bacteria.

For example, the bacteria Pseudomonas aeruginosa is widely distributed in water, and also in soil, sewage and plants. Pseudomonas aeruginosa use acylated homoserine lactones (HSL) as signals to control various functions of their communal environment, e.g., to control biofilms harboring them. One identified HSL signal for Pseudomonas aeruginosa is

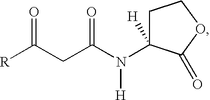

which represents an “attachment” signal for Pseudomonas aeruginosa when R is a propyl group. Another identified HSL signal for Pseudomonas aeruginosa is:

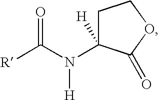

which represents a “detachment” signal or Pseudomonas aeruginosa when R′ is a propyl group.

Biofilms can develop into macroscopic structures several millimeters or centimeters in thickness and cover large surface areas. For non-living objects, these formations can play a role in restricting or entirely blocking flow in plumbing system decreasing heat transfer in heat exchangers, or causing pathogenic problems in municipal water supplies, food processing, medical devices (e.g., catheters, orthopedic devices, implants). Moreover, biofilms often decrease the life of materials through corrosive action mediated by the embedded microorganisms. This biological fouling is a serious economic problem in industrial water process systems, pulp and paper production processes, cooling water systems, injection wells for oil recovery, cooling towers, porous media (sand and soil), marine environments, and air conditioning systems, and any closed water recirculation system. Biofilms are also a severe problem in medical science and industry, said to cause dental plaque, infections, contaminated endoscopes and contact lenses, prosthetic device colonisation and biofilm formation on medical implants.

In other areas, biofilms can be quite useful. For example, biofilms are an integral part of the human body and are especially beneficial in protecting the intestine from attack by harmful bacteria. One of the best examples of beneficial application of biofilms to solve a major problem is in the cleaning of wastewater. Biofilms can also be used to clean up oil and gasoline spills. Bioremediation using biofilms has emerged as optional technology for cleaning up groundwater at many sites containing hazardous waste.

Numerous publications describe compositions and methods directed as treating water in an effort to destroy biofilms and the bacteria they harbor. Dimethylhydantoin (DMH) is used in some halogenated biocidal water treatment products. U.S. Pat. No. 6,638,959 describes the use of dibromodialkylnydantoin (DBDAH), e.g., dibromodimethylhydantoin (DBDMH) to water that is in contact with or that comes into contact with, biofilms.

In spite of the current body of knowledge regarding biofilms and cell-to-cell communication, there remains a need for commercially feasible means of utilizing cell-to-cell communication to control biofilms harboring bacteria and other contaminants, whether the contaminants originated in water or some other medium. Additionally, in spite of the availability of commercial halogenated biocides, there remains a need for improved halogenated biocides with enhanced biofilm controlling capabilities.

### THE INVENTION

This invention meets the above-described needs by providing

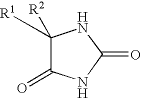

in contact biofilm comprising a microorganism, wherein

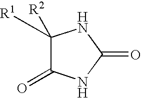

emulates a cell-to-cell signal molecule of the microorganism, R¹ comprises a hydrogen atom or a hydrocarbyl radical having from 1 to about 14 carbon atoms and R² in comprises a hydrocarbyl radical having from 1 to out 14 carbon atoms. Molecules of this invention are designed to be physically and electrically similar to cell-to-cell signal molecules for microorganisms such that the molecules of this invention emulate the cell-to-cell signal molecules. Also provided methods for synthesizing

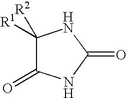

suitable for controlling, biofilm, wherein R¹ comprises a hydrogen atom or a hydrocarbyl radical having from 1 to about 14 carbon atoms and R² independently comprises a hydrocarbyl radical having from 1 to about 14 carbon atoms, the methods comprising converting a ketone to a cyanohydrin by combining the ketone with sodium bisulfate, sodium cyanide and ammonium carbonate. Also provided are methods for controlling biofilm on substree in contact with liquid medium, wherein the biofilm comprises a microorganism the methods comprising introducing into the liquid medium

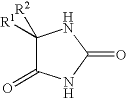

that emulates a cell-to-cell signal molecule of the microorganism wherein R¹ comprises a hydrogen atom or a hydrocarbyl radical having from 1 to about 14 carbon atoms and R² independently comprises a hydrocarbyl radical having from 1 to about 14 carbon atoms. R¹ and R² can be the same or different. In some embodiments, when R¹ and/or R² comprise a hydrocarbyl radical, the radical has from 1 to about 6, or 3 to 6, carbon atoms.

As used herein: (i) “biofilm” means a mucilaginous community that can grow on various substrates, said community comprised of one or more microorganisms, such as, for example, bacteria, archaea, fungi, molds, algae or protozoa, or the like; (ii) “substrate” means any surface on which a biofilm can form or has formed, and includes, but is not limited to, hard or soft surfaces such as polymers, plastics, tubing, ceramics, metals, glass, hydroxyapatite, skin, bone, tissues, and geological formations such as layers of sand; (iii) “to control/controlling” a biofilm means to cause dissolution of at least a portion of a biofilm, to prevent formation of a biofilm or additional growth of an existing biofilm, and/or to cause a biofilm to form in a more desired manner or location. Cell-to-cell signal molecules for microorganisms comprising a biofilm control the biofilm by sending appropriate signals. For example, a “detachment” signal of a microorganism signals the microorganism to detach from a biofilm and may cause dissolution of at least a portion of the biofilm; a “do not attach” signal or “remain planktonic” signal of a microorganism signals the microorganism to remain planktonic and may prevent formation of a biofilm or additional growth of an existing biofilm; an “attachment” signal of a microorganism signals the microorganism to attach, e.g., to a biofilm, and given at appropriate times and locations may cause a biofilm to form in a more desired manner or location; and a “do not detach” signal or “remain sessile” signal of a microorganism signals a microorganism to remain sessile and may prevent at least a portion of a biofilm from dissolving.

This invention also provides

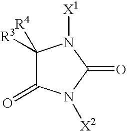

in contact with biofilm comprising a microorganism, wherein

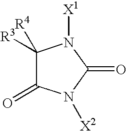

emulates a cell-to-cell signal molecule of the microorganism, R³ comprises a hydrogen atom or a hydrocarbyl radical having from 1 to about 14 carbon atoms, R⁴ independently comprises a hydrocarbyl radical having from 1 to about 14 carbon atoms, and either (i) X¹ comprises a hydrogen atom or a halogen atom and X² independently comprises a halogen atom, or (ii) X² comprises a hydrogen atom or a halogen atom and X¹ independently comprises a halogen atom. This embodiment of this invention is directed toward novel

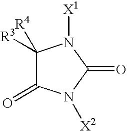

having combinations of R³, R⁴, X¹ and X² that have heretofore not been known to emulate cell-to-cell signal molecules for microorganisms; and dibromodialkylhydantoins (DBDAH) and bromochlorodialkylhydantoins (BCDAH) are excluded from this embodiment of this invention. Also provided are methods for synthesizing

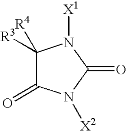

suitable for controlling biofilm, wherein R³ comprises a hydrogen atom or a hydrocarbyl radical having from 1 to about 14 carbon atoms, R⁴ independently comprises a hydrocarbyl radical having from 1 to about 14 carbon atoms, and either (i) X¹ comprises a hydrogen atom or a halogen atom and X² independently comprises a halogen atom, or (ii) X² comprises a hydrogen atom or a halogen atom and X¹ independently comprises a halogen atom, the methods comprising converting a ketone to a cyanohydrin by combining the ketone with sodium bisulfate, sodium cyanide and ammonium carbonate. Also provided are methods for controlling biofilm on substrate in contact with aqua medium, wherein the biofilm comprises a microorganism, comprising introducing into the liquid medium

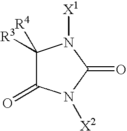

according to this embodiment that emulates a cell-to-cell signal molecule of the microorganism, wherein R³ comprises a hydrogen atom or a hydrocarbyl radical having from 1 to about 14 carbon atoms, R⁴ independently comprises a hydrocarbyl radical having from 1 to about 14 carbon atoms, and either (i) X¹ comprises a hydrogen atom or a halogen atom and X² independently comprises a halogen atom, or (ii) X² comprises a hydrogen atom or a halogen atom and X¹ independently comprises a halogen atom. R³ and R⁴ can be the same or different, in some embodiments, when R³ and/or R⁴ comprise a hydrocarbyl radical, the radical has from 1 to about 6, or 3 to 5, carbon atoms. X¹ and X² can be the same or different.

This invention also provides compositions in contact with biofilm comprising a microorganism, the compositions comprising a molecule capable of killing the microorganism and

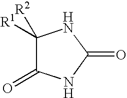

that emulates a cell-to-cell signal molecule of the microorganism, wherein R¹ comprises a hydrogen atom or a hydrocarbyl radical having from 1 to about 14 carbon atoms and R² independently comprises a hydrocarbyl radical having from 1 to about 14 carbon atoms. As used in this embodiment, the molecule capable of killing bacteria can comprise any molecule capable of killing the bacteria, including without limitation dibromodialkylhydantoins (DBDAH), bromochlorodialkylhydantoins (BCDAH), and

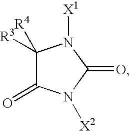

wherein R³ comprises a hydrogen atom or a hydrocarbyl radical having from 1 to about 14 carbon atoms, R⁴ independently comprises a hydrocarbyl radical having from 1 to about 14 carbon atoms, and either (i) X¹ comprises a hydrogen atom or a halo en atom and X² independently comprises a halogen atom, or (ii) X² comprises a hydrogen atom or a halogen atom and X¹ independently comprises a halogen atom. Also provided are methods for killing a microorganism such as bacteria, at least a portion of which is harbored in biofilm on substrate in contact with liquid medium, comprising introducing into the liquid medium at least one molecule capable of killing the microorganism and

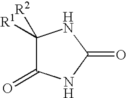

that emulates a cell-to-cell signal molecule of the microorganism, wherein R¹ comprises a hydrogen atom or a hydrocarbyl radical having from 1 to about 14 carbon atoms and R² independently comprises a hydrocarbyl radical having from 1 to about 14 carbon atoms. Each of R¹, R², R³ and R⁴ can be the same or different.

This invention is described in connection with specific embodiments. It is understood that this invention is not limited to any one of these specific embodiments.

In

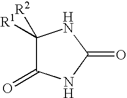

according to this invention, R¹ and R² are independently selected to have the length and electronics needed to enable the molecule to emulate the desired cell-to-cell signal molecule. For purposes of example only, either or both of R¹ and R² can comprise a carbonyl or an alkyl.

### In

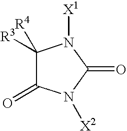

according to this invention, R³ and R⁴ are independently selected to have the length and electronics needed to enable the molecule to emulate the desired cell-to-cell signal molecule. For purposes of example only, either or both of R³ and R⁴ can comprise a carbonyl or an alkyl. When the molecule is intended to kill bacteria, either or both of X¹ and X² can comprise a halogen atom capable of killing the bacteria, including without limitation a bromine atom, a chlorine atom, or an iodine atom.

In

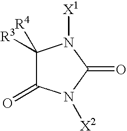

according to this invention, the hydantoin nucleus retains the ability to stabilize the halogen atom(s) X¹ and/or X² attached to the N atoms within the ring. Depending on the composition of X¹, X², R³, and R⁴, this stabilization can be increased or decreased. When either or both of X¹ and X² is a bromine, the product bromamine is not only capable of holding the halogen and stabilizing it, but it is also capable of releasing it as active HOBr in aqueous solution according to the following acid-based equilibrium reaction:

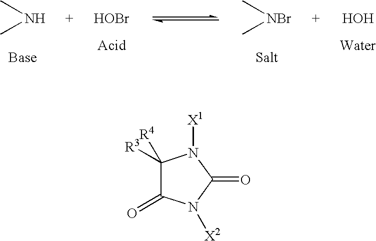

according to this invention may be synthesized by converting a ketone molecule to a molecule having the same ligands groups on the 5-carbon, as were initially present as flanking groups at the ketone carbonyl. Other routes are also possible and should not be considered to be excluded from this invention.

### EXAMPLES

In the molecules of this invention illustrated in the following examples, R¹ and R² were selected for size, length, and electronic character, to make molecules that are essentially indistinguishable from legitimate bacterial molecular signals.

### Example 1

### 5-Methyl-5-Ethylhydantoin

2-Butanone was converted to an intermediate cyanohydrin (I) by treatment with sodium bisulfite followed by sodium cyanide. The intermediate cyanohydrin (I) was admixed with ammonium carbonate and warmed to form the crude 5-methyl-5-ethylhydantoin (II), which was isolated and purified by standard crystallization and re-crystallization operations.

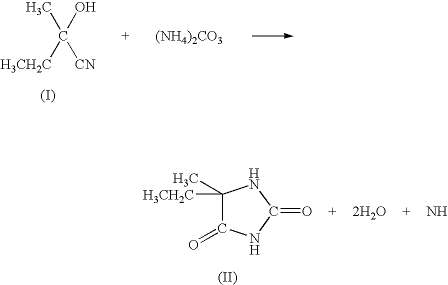

### Example 2

### 5-Methyl-5-Propylhydantoin

Using a method similar to that used in Example 1, 2-Pentanone was converted to an intermediate cyanohydrin (III) and finally to the desired 5-methyl-5-propylhydantoin (IV).

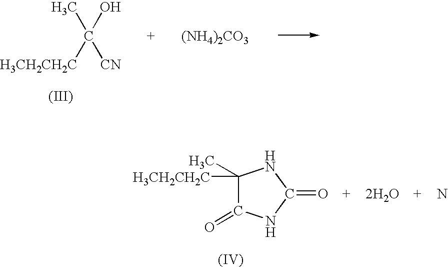

### Example 3

### 5,5-Diethylhydantoin

Using a method similar to that used in Example 1, 1,3-Pentanone was converted to a symmetric, intermediate cyanohydrin and finally to the desired 5,5-diethylhydantoin.

### Example 4

### 5-Methyl-5-Heptylhydantoin

Using a method similar to that used in Example 1, 2-Nonanone was converted to the desired product 5-methyl-5-heptylhydantoin.

### Example 5

### 5-(1,1,1-trifluoromethyl)-5-methylhydantoin

Using a method similar to that used in Example 1, an intermediate 1,1,1-trifluoroacetone cyanohydrin was converted to, 5-(1,1,1-trifluoromethyl)-5-methylhydantoin (V), when R³ is a normal methyl group.

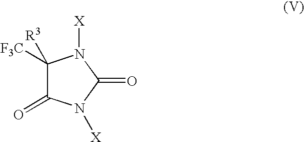

Example 5 illustrates that this invention includes the incorporation of fluorinated alkyl groups, such as the trifluormethyl substituent shown, or other, even more complex modifications. It is believed that the presence of profoundly electronegative groups the ligands of the substituted hydantoin will have equally striking pertubations on the type of signal being relayed in the treated biologic systems.

Compositions according to this invention have the ability to behave as oxidizing biocides, in their own right. For example, bromoamines are biocidal. Other embodiments, such as chloramines, while not nearly as effective biocides as bromoamines, are still irritants to bacteria, and under some condition may also provide biocidal action. Chlorinated or mixed chlorine and other halogen species are expected to provide slow, or long-term, residual biocidal control effects.

While the present invention has been described in terms of one or more preferred embodiments, it is to be understood that other modifications may be made without departing from the scope of the invention, which is set forth in the claims below.

We claim:

1. 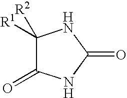
in contact with biofilm comprising a microorganism, wherein 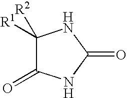
emulates a cell-to-cell signal molecule of the microorganism, R¹ comprises a hydrogen atom or a hydrocarbyl radical having from 1 to about 14 carbon atoms and R² independently comprises a hydrocarbyl radical having from 1 to about 14 carbon atoms.

2. A method for controlling biofilm on substrate in contact with liquid medium, wherein the biofilm comprises a microorganism, the method comprising introducing into the liquid medium at least one 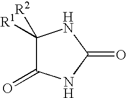
that emulates a cell-to-cell signal molecule of the microorganism, wherein R¹ comprises a hydrogen atom or a hydrocarbyl radical haying from 1 to about 14 carbon atoms and R² independently comprises a hydrocarbyl radical having from 1 to about 14 carbon atoms.

3. The method of claim 2 wherein the 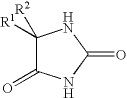
emulates a cell-to-cell signal molecule that signals the microorganism to detach from the biofilm.

4. The method of claim 2 wherein the 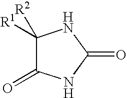
emulates a cell-to-cell signal molecule that signals the microorganism to attach to the biofilm.

5. The method of claim 2 wherein the 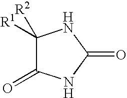
emulates a cell-to-cell signal molecule that signals the microorganism to remain planktonic.

6. The method of claim 2 wherein the 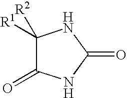
emulate a cell-to-cell signal molecule that signals the microorganism to remain sessile.

7. A composition in contact with biofilm comprising one or more microorganisms wherein the composition comprises (i) 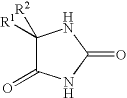
that emulates a cell-to-cell signal molecule of one of the microorganisms, wherein R¹ comprises a hydrogen atom or a hydrocarbyl radical having from 1 to about 14 carbon atoms and R² independently comprises a hydrocarbyl radical having from 1 to about 14 carbon atoms, and (ii) 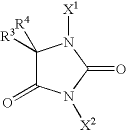
that emulates a cell-to-cell signal molecule of one of the microorganisms, wherein R³ comprises a hydrogen atom or a hydrocarbyl radical having from 1 to about 14 carbon atoms, R⁴ independently comprises a hydrocarbyl radical having from 1 to about 14 carbon atoms, and either (i) X¹ comprises a hydrogen atom or a halogen atom and X² independently comprises a halogen atom, or (ii) X² comprises a hydrogen atom or a halogen atom and X¹ independently comprises a halogen atom.

8. A method for controlling biofilm on substrate in contact with liquid medium, wherein the biofilm comprises one or more microorganisms, the method comprising introducing into the liquid medium at least one 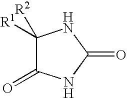
that emulates a cell-to-cell signal molecule of one of the microorganisms wherein R¹ comprises a hydrogen atom or a hydrocarbyl radical having from 1 to about 14 carbon atoms and R² independently comprises a hydrocarbyl radical having from 1 to about 14 carbon atoms, and at least one 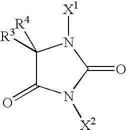
that emulates a cell-to-cell signal molecule of one of the microorganisms, wherein R³ comprises a hydrogen atom or a hydrocarbyl radical haying from 1 to about 14 carbon atoms, R⁴ independently comprises a hydrocarbyl radical having from 1 to about 14 carbon atoms and either (1) X¹ comprises a hydrogen atom or a halogen atom and X² independently comprises a halogen atom, or (ii) X⁴ comprises a hydrogen atom or a halogen atom and X¹ independently comprises a halogen atom.

9. A composition in contact with biofilm comprising a microorganism, the composition comprising a molecule capable of killing the microorganism and 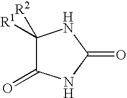
that emulates a cell-to-cell signal molecule of the microorganism, wherein R¹ comprises a hydrogen atom or a hydrocarbyl radical having from 1 to about 14 carbon atoms and R² independently comprises a hydrocarbyl radical having from 4 to about 14 carbon atoms.

10. A method for killing a microorganism, at least a portion of which is harbored in biofilm on substrate in contact with liquid medium, comprising introducing into the liquid medium at least one molecule capable of killing the microorganism and 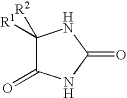
that emulates a cell-to-cell signal molecule of the microorganism, wherein R¹ comprises a hydrogen atom or a hydrocarbyl radical having from 1 to about 14 carbon atoms and R² independently comprises a hydrocarbyl radical having from 1 to about 14 carbon atoms.
